(19) United States
(12) Patent Application Publication
(10) Pub. No.: US 2010/0160462 A1
(43) Pub. Date: Jun. 24, 2010

(54) HIGH-SPEED STOP IN A FISCHER-TROPSCH PROCESS

(76) Inventors: Thomas Joris REMANS, Amsterdam (NL); Wilhelmus Johannes Franciscus SCHOLTEN, Education City (QA)

Correspondence Address: SHELL OIL COMPANY, P O BOX 2463, HOUSTON, TX, 772522463, US

(21) Appl. No.: 12/638,664

(22) Filed: Dec. 15, 2009

(30) Foreign Application Priority Data
Dec. 16, 2008 (EP) 08171744.9

(51) Int. Cl. C07C 27/06

(52) U.S. Cl. 518/712

(57) ABSTRACT

A process for carrying out a high-speed stop in a Fischer-Tropsch process which comprises providing a feed comprising CO and H₂ to a fixed bed reactor comprising a Fischer-Tropsch catalyst, the reactor being at reaction temperature and pressure, the gaseous hourly space velocity in the reactor being in the range from 500 to 10000 Nl/l/h, and withdrawing an effluent from the reactor, wherein the high-speed stop is effected by blocking provision of H₂ to the reactor while providing

CO to the reactor, and withdrawing gaseous reactor content from the reactor, and wherein during the high-speed stop CO is added in an amount of 5-80 Nl/l/h.

This application claims the benefit of European Application No. 08171744.9 filed Dec. 16, 2008 which is incorporated herein by reference.

### BACKGROUND OF THE INVENTION

The present invention relates to a Fischer-Tropsch process, in particular to a process for carrying out a high-speed stop in a Fischer-Tropsch process carried out in a fixed bed reactor.

The Fischer-Tropsch process can be used for the conversion of hydrocarbonaceous feed stocks into normally liquid and/or solid hydrocarbons (0° C., 1 bar). The feed stock (e.g. natural gas, associated gas, coal-bed methane, residual oil fractions, biomass and/or coal) is converted in a first step into a mixture of hydrogen and carbon monoxide. This mixture is often referred to as synthesis gas or syngas. The synthesis gas is fed into a reactor where it is converted over a suitable catalyst at elevated temperature and pressure into paraffinic compounds ranging from methane to high molecular weight molecules comprising up to 200 carbon atoms, or, under particular circumstances, even more.

Numerous types of reactor systems have been developed for carrying out the Fischer-Tropsch reaction. For example, Fischer-Tropsch reactor systems include fixed bed reactors, especially multi-tubular fixed bed reactors, fluidised bed reactors, such as entrained fluidised bed reactors and fixed fluidised bed reactors, and slurry bed reactors such as three-phase slurry bubble columns and ebullated bed reactors.

The Fischer-Tropsch reaction is very exothermic and temperature sensitive. In consequence, careful temperature control is required to maintain optimum operation conditions and desired hydrocarbon product selectivity.

The fact that the reaction is very exothermic also has the consequence that when temperature control is not adequate, the reactor temperature can increase very quickly, which carries the risk of a reactor runaway, which may result in local deactivation of the catalyst.

In GB-2246576-A a high-speed stop process is described for a fixed bed Fischer-Tropsch reactor. The supply of carbon monoxide and hydrogen is interrupted. In a later step hydrogen is supplied to protect the catalyst.

The desired use of high-activity catalysts in Fischer-Tropsch fixed-bed reactors makes the situation even more challenging, because the susceptibility of a reactor to reactor runaway increases with increased catalyst activity. A reactor runaway is a most undesirable phenomenon, as it may result in catalyst deactivation which necessitates untimely replacement of the catalyst, causing reactor downtime and additional catalyst cost.

Therefore, there is need for an improved process for carrying out a high-speed stop in a fixed bed Fischer-Tropsch reactor. A high-speed stop may, for example, be required when the temperature in the Fischer-Tropsch reactor increases to an unacceptable value either locally or over the entire reactor, when there is an interruption in the gas flow, or in the case of other unforeseen circumstances.

### SUMMARY OF THE INVENTION

Accordingly, the present invention pertains to a process for carrying out a high-speed stop in a Fischer-Tropsch process which comprises providing a feed comprising CO and H₂ to a fixed bed reactor comprising a Fischer-Tropsch catalyst, the reactor being at reaction temperature and pressure, the gaseous hourly space velocity in the reactor being in the range from 500 to 10000 Nl/l/h, and withdrawing an effluent from the reactor; wherein the high-speed stop is effected by blocking provision of H₂ to the reactor while providing CO to the reactor, and withdrawing gaseous reactor content from the reactor; and wherein during the high- speed stop CO is added in an amount of 5-80 Nl/l/h.

### DETAILED DESCRIPTION OF THE INVENTION

A process according to the present invention may be referred to as “AL Kinar”-process.

Depending on the process configuration, the performance of a high-speed stop in a fixed bed Fischer-Tropsch reactor is often accompanied by a rise in temperature, culminating in a process-side temperature peak. This is caused by a decrease in gas hourly space velocity which leads to an increased conversion, accompanied by increased heat formation, and simultaneously to a decrease in heat removal capacity.

It was found that a high-speed stop according to the invention, where CO is provided to the fixed bed reactor during the high-speed stop, results in an increase in peak temperature which is substantially lower than the increase in peak temperature which is obtained when a high-speed stop is carried out by blocking the flow of feed to the reactor, whether or not accompanied by the provision on an inert feed. It was also found that the procedure according to the invention does not result in substantial catalyst deactivation.

Not wishing to be bound by theory it is believed that the CO added during the high-speed stop decreases the activity of the catalyst, and thus lessens the exotherm produced by the reaction. If this mechanism is correct, it is surprising that the procedure according to the invention does not result in substantial catalyst deactivation.

In order to restart the Fischer-Tropsch process after a high-speed stop in accordance to the present invention, one may subject the catalyst to syngas. If desired, one may after a high-speed stop in accordance to the present invention, subject the catalyst first to hydrogen and then to syngas in order to restart the Fischer-Tropsch process.

The invention will be described in more detail below.

In the process of the invention, the provision of H2 to the reactor is stopped. Where the feed comprising CO and H2 to the reactor comprises a mixture of CO and H2, e.g., in the form of a syngas feed, the stopping of the H2 feed will in effect be the stopping of the mixture. In that case the CO provided during the high-speed stop will be provided as a separate feed. Where the feed to the reactor comprises H2 and CO provided through different feed streams it is in principle possible to stop only the H2 feed, and allow the CO feed to continue. However, for reasons of process reliability, it is considered preferred also in that case to have the possibility to provide CO from a different source during the high-speed stop, for the case that the regular CO supply is compromised.

During the high-speed stop, the CO is generally provided in an amount of at least 5 Nl/l/h, more in particular at least 10 Nl/l/h, still more in particular at least 20 Nl/l/h. It was found that if the amount of CO added is too low, the advantageous effects of the present invention will not be obtained. The CO is generally provided in an amount of at most 80 Nl/l/h, more in particular at least 70 Nl/l/h, still more in particular at least 60 Nl/l/h. It was found that the addition of too much CO will not contribute further to controlling the temperature peak while it may contribute to catalyst deactivation.

The CO added in the process according to the invention may be added in many forms. In one embodiment the CO is provided from a storage vessel containing a CO-containing gas with a CO content of at least 80 mol. %, in particular at least 90 mol. %, still more in particular at least 95 mol. %, even more in particular at least 98 mol. % of CO. The balance will consist of a gas which is inert under Fischer-Tropsch reaction conditions. Examples of suitable inerts include nitrogen and low-sulphur natural gas, for example desulphurised natural gas.

In one embodiment the addition of CO is accompanied by the addition of inert gas, pre-mixed with the CO, or separately. Inert gas may be added, for example in an amount of at least 5 Nl/l/h, more in particular at least 10 Nl/l/h, still more in particular at least 20 Nl/l/h. The amount of inert gas, if added, may for example be at most 80 Nl/l/h, more in particular at least 70 Nl/l/h, still more in particular at least 60 Nl/l/h.

On the one hand the addition of inert gas may serve to help control the formation of an exotherm. On the other hand, the addition of inert gas may dilute the effect of the CO. On the basis of the above, it is within the scope of the skilled person to determine whether, and if so, how much, inert gas is added during the high-speed stop.

During the high-speed stop gaseous reactor content is withdrawn from the reactor. This reactor content encompasses gaseous reactants, gaseous products, and any inert gases added to the reactor during the reaction or during the high-speed stop. Depending on the reactor configuration liquid reaction products present in the unit may or may not be withdrawn from the reactor during the high-speed stop.

The Fischer-Tropsch reactor comprises a catalyst section located between the inlet section of the reactor and the outlet section of the reactor. The inlet section of the reactor is provided with an inlet for the reactants, viz. hydrogen and CO, for CO during the high-speed stop, and optionally for inert gas to be added during the reaction or during the high-speed stop. As will be evident to the skilled person, the various components can be added to the reactor though the same or different inlets, depending on reactor configuration. The outlet section of the reactor is provided with an outlet for liquid product and an outlet for gaseous reactor content. Depending on reactor configuration, these outlets may be combined, or provided separately.

In the context of the present specification the wording bottom of the reactor refers to the part of the reactor below the part of the reactor where the catalyst is located. The wording top of the reactor refers to the part of the reactor above the part of the reactor where the catalyst is located. In the Fischer-Tropsch process according to the invention, the inlet section is generally provided in the top of the reactor. The outlet section is generally provided in the bottom of the reactor.

The withdrawal of gaseous reactor content during the high-speed stop results in a reduction of the pressure in the reactor. The final pressure that is obtained is, generally, below 15 bar, more specifically in the range of 1-10 bar, for example in the range of 2-8 bar. The amount of reactor content removed during the high-speed stop is determined to a large extent by the desired pressure to be obtained.

The reactor is generally operated before the high-speed temperature stop at an operating pressure which generally ranges from 5 to 150 bar, preferably from 20 to 80 bar, more in particular from 30 to 70 bar.

The provision of CO in the high-speed stop can be stopped when the exotherm is under control, in other words, when the reactor temperature reaches a value within normal operation ranges. Depending on the speed with which the reactor is depressurised, it will typically be possible to stop the provision of CO-containing gas after 2-20 minutes, more specifically after 5-10 minutes. Leaving the CO flow on for longer is also possible if that is desired.

Depending on the design of the reactor, the effluent from the reactor during operation can be a single gaseous phase, a multi-phase effluent or two effluent streams with one being mainly gaseous and one being mainly liquid phase.

If so desired, the reactor may be cooled during or after the high-speed stop. It is preferred to cool the reactor during the high speed stop. The end temperature of the cooling step depends on the desired further action. In general, the reactor will be cooled to a temperature between ambient and 200° C. Where the reactor is cooled with a view to immediate restarting of the reactor, it will generally be cooled to a temperature in the range of 100-190° C., in particular to a value of 160-180° C.

The cooling speed will depend on the size of the reactor and further circumstances. For example, it may be in the range of 10-100° C. per hour.

The process according to the invention is suitable for fixed bed reactors. In a preferred embodiment the reactor is a reactor tube, which has a ratio between length and diameter of at least 5, in particular at least 50. As an upper limit a ratio of at most 1000 may be mentioned.

In one embodiment, the reactor tube is a tube in a multitubular reactor, which comprises a plurality of reactor tubes at least partially surrounded by a heat transfer medium.

The tubes in a multitubular reactor generally have a diameter in the range of 0.5-20 cm, more in particular in the range of 1 to 15 cm. They generally have a length in the range of 3 to 30 m. The number of tubes in a multitubular reactor is not critical to the present invention and may vary in wide ranges, for example in the range of 4 to 50 000, more in particular in the range of 100 to 40 000.

Multitubular reactors and their use in Fischer-Tropsch processes are known in the art and require no further elucidation here.

In one embodiment, the catalyst is a particulate catalyst, that is, a catalyst in the form of particles. The shape of the catalyst may be regular or irregular. The dimensions are suitably 0.1-30 mm in all three directions, preferably 0.1-20 mm in all three directions, more preferably 0.5-20 mm, more in particular 0.1-6 mm, even more preferably 0.5-6 mm in all three directions. Suitable shapes are spheres and, in particular, extrudates. The extrudates suitably have a length between 0.5 and 30 mm, preferably between 1 and 6 mm. The extrudates may be cylindrical, polylobal, or have any other shape. Their effective diameter, that is, the diameter of a sphere with the same outer surface over inner volume ratio, is suitably in the range of 0.1 to 10 mm, more in particular in the range of 0.2-6 mm.

A fairly recent trend in the development of Fischer-Tropsch catalysts is the development of catalyst particles with a decreased diffusion limitation. It has been found that catalysts with a decreased diffusion limitation are highly active in Fischer-Tropsch processes. However, due to their high activity, and their higher activation energy, their use entails an increased risk of reactor runaway. Further, it has also been found that catalysts with a decreased diffusion limitation are particularly sensitive to how a high-speed stop is carried out. More in particular, it has been found that for a catalyst with a decreased diffusion limitation a high-speed stop performed by blocking the flow of feed to the reactor and depressurising the reactor via the bottom may lead to the formation of a temperature peak of the order of 100° C., which is difficult to address in commercial operation. On the other hand, when for the same decreased diffusion limitation catalyst the high-speed stop according to the invention was carried out, this resulted in an increase in peak temperature on the order of 23° C. Therefore, the process according to the invention is of particular interest for reactors comprising a catalyst with decreased diffusion limitation, in particular with an effective diameter of at most 2 mm, more in particular of at most 1.6 mm, still more in particular of at most 1.5 mm. Catalysts with a decreased diffusion limitation are for example described in WO2003/013725, WO2008/087149, WO2003/103833, and WO2004/041430.

The Fischer-Tropsch reaction is preferably carried out at a temperature in the range from 125 to 400° C., more preferably 175 to 300° C., most preferably 200 to 260° C. The gaseous hourly space velocity may vary within wide ranges and is typically in the range from 500 to 10000 Nl/l/h, preferably in the range from 700 to 4500 Nl/l/h, more preferably in the range from 1500 to 4000 Nl/l/h. The hydrogen to CO ratio of the feed as it is fed to the catalyst bed generally is in the range of 0.5:1 to 2:1. As indicated above, in one embodiment the feed is provided to the reactor in the form of a mixture of hydrogen and CO, for example in the form of a syngas feed. In another embodiment, the hydrogen and CO are provided to the reactor in different streams.

Products of the Fischer-Tropsch synthesis may range from methane to heavy hydrocarbons. Preferably, the production of methane is minimized and a substantial portion of the hydrocarbons produced have a carbon chain length of a least 5 carbon atoms. Preferably, the amount of C5+ hydrocarbons is at least 60% by weight of the total product, more preferably, at least 70% by weight, even more preferably, at least 80% by weight, most preferably at least 85% by weight. The CO conversion of the overall process is preferably at least 50%.

The products obtained via the process according to the invention can be processed through hydrocarbon conversion and separation processes known in the art to obtain specific hydrocarbon fractions. Suitable processes are for instance hydrocracking, hydroisomerisation, hydrogenation and catalytic dewaxing. Specific hydrocarbon fractions are for instance LPG, naphtha, detergent feedstock, solvents, drilling fluids, kerosene, gasoil, base oil and waxes.

Fisher-Tropsch catalysts are known in the art. They typically comprise a Group VIII metal component, preferably cobalt, iron and/or ruthenium, more preferably cobalt. Typically, the catalysts comprise a catalyst carrier. The catalyst carrier is preferably porous, such as a porous inorganic refractory oxide, more preferably alumina, silica, titania, zirconia or combinations thereof. References to the Periodic Table and groups thereof used herein refer to the previous IUPAC version of the Periodic Table of Elements such as that described in the 68th Edition of the Handbook of Chemistry and Physics (CPC Press).

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material.

The catalytically active metal may be present in the catalyst together with one or more metal promoters or co-catalysts. The promoters may be present as metals or as the metal oxide, depending upon the particular promoter concerned. Suitable promoters include oxides of metals from Groups IA, IB, IVB, VB, VIB and/or VIIB of the Periodic Table, oxides of the lanthanides and/or the actinides. Preferably, the catalyst comprises at least one of an element in Group IVB, VB and/or VIIB of the Periodic Table, in particular titanium, zirconium, manganese and/or vanadium. As an alternative or in addition to the metal oxide promoter, the catalyst may comprise a metal promoter selected from Groups VIIB and/or VIII of the Periodic Table. Preferred metal promoters include rhenium, platinum and palladium.

A most suitable catalyst comprises cobalt as the catalytically active metal and zirconium as a promoter. Another most suitable catalyst comprises cobalt as the catalytically active metal and manganese and/or vanadium as a promoter. The promoter, if present in the catalyst, is typically present in an amount of from 0.1 to 60 parts by weight per 100 parts by weight of carrier material. It will however be appreciated that the optimum amount of promoter may vary for the respective elements which act as promoter. If the catalyst comprises cobalt as the catalytically active metal and manganese and/or vanadium as promoter, the cobalt : (manganese+vanadium) atomic ratio is advantageously at least 12:1.

It will be understood that it is within the scope of the skilled person to determine and select the most appropriate conditions for a specific reactor configuration and reaction regime.

The present invention is illustrated by the following examples, without being limited thereto or thereby.

### Comparative Example

A Fischer-Tropsch process was operated at a temperature of about 210° C. and a pressure of about 40 bar in a fixed-bed reactor containing a catalyst.

A high-speed stop was carried out by blocking the flow of feed to the reactor, while maintaining a nitrogen feed at an LHSV of 50 Nl/l/h. The reactor was depressurised via the bottom to a pressure of 20 barg in 6 minutes, and then to a pressure of 6 barg in an additional 14 minutes. The reactor temperature was measured during the high-speed stop, and a peak temperature of +100° C. above the maximum reaction temperature prior to the high-speed stop was measured.

After the high-speed stop, the catalyst in the reactor was subjected to hydrogen, and in a next step to syngas. Upon restart of the reactor it was found that no catalyst deactivation had taken place.

### Example According to the Invention

A Fischer-Tropsch process was operated at a temperature of about 210° C. and a pressure of about 40 bar in a fixed-bed reactor containing a catalyst.

A high-speed stop was carried out by blocking the flow of feed to the reactor, and adding a CO feed at an LHSV of 42 Nl/l/h. The reactor was depressurised via the bottom to a pressure of 20 barg in 6 minutes, and then to a pressure of 6 barg in an additional 14 minutes. The reactor temperature was measured during the high-speed stop, and a peak temperature of +57° C. above the maximum reaction temperature prior to the high-speed stop was measured.

After the high-speed stop, the catalyst in the reactor was subjected to hydrogen, and in a next step to syngas. Upon restart of the reactor it was found that no catalyst deactivation had taken place.

### Comparison of the Experimental Data

In the comparative example, the reactor temperature was measured during the high-speed stop, and a peak temperature of +100° C. above the maximum reaction temperature prior to the high-speed stop was measured.

In the example according to the current invention, the reactor temperature was measured during the high-speed stop, and a peak temperature of +57° C. above the maximum reaction temperature prior to the high-speed stop was measured.

Hence, it appeared that the procedure according to the invention resulted in a substantially lower peak temperature as compared to the procedure in the Comparative Example.

What is claimed is:

1. A process for carrying out a high-speed stop in a Fischer-Tropsch process which comprises providing a feed comprising CO and H₂ to a fixed bed reactor comprising a Fischer-Tropsch catalyst, the reactor being at reaction temperature and pressure, the gaseous hourly space velocity in the reactor being in the range from 500 to 10000 Nl/l/h, and withdrawing an effluent from the reactor;
wherein the high-speed stop is effected by blocking provision of H₂ to the reactor while providing CO to the reactor, and withdrawing gaseous reactor content from the reactor;
and wherein during the high-speed stop CO is added in an amount of 5-80 Nl/l/h.

2. A process according to claim 1, wherein the gaseous reactor content is removed to reduce the reactor pressure to a value below 15 bar.

3. A process according to claim 1, wherein the CO provided during the high-speed stop originates from a source different from the feed comprising CO and H₂.

4. A process according to claim 1, wherein liquid reaction product is retained in the unit during the high-speed stop.

5. A process according to claim 1, wherein liquid reaction product is removed from the reactor during the high-speed stop.

6. A process according to claim 1, wherein the provision of CO is stopped after 2-20 minutes.

7. A process according to claim 1, wherein the CO is provided from a storage vessel containing a CO-containing gas with a CO content of at least 80 mol. %, the balance consisting of a gas which is inert under Fischer-Tropsch reaction conditions.

8. A process according to claim 1, wherein the reactor is a reactor tube with a ratio between length and diameter of at least 50:1.

9. A process according to claim 8, wherein the reactor tube is a tube in a multitubular reactor, which comprises a plurality of reactor tubes at least partially surrounded by a heat transfer medium.

10. A process according to claim 1, wherein the Fischer-Tropsch catalyst is a particulate catalyst with an effective diameter of at most 1.6 mm, in particular at most 1.5 mm.

11. A process for carrying out a high-speed stop in a Fischer-Tropsch process which comprises providing a feed comprising CO and H₂ to a fixed bed reactor comprising a Fischer-Tropsch catalyst, the reactor being at reaction temperature and pressure, the gaseous hourly space velocity in the reactor being in the range from 500 to 10000 Nl/l/h, and withdrawing an effluent from the reactor;
wherein the high-speed stop is effected by blocking provision of H₂ to the reactor while providing CO to the reactor, and withdrawing gaseous reactor content from the reactor;
wherein during the high-speed stop CO is added in an amount of 5-80 Nl/l/h; and
wherein the CO provided during the high-speed stop originates from a source different from the feed comprising CO and H₂.

12. A process according to claim 11, wherein the gaseous reactor content is removed to reduce the reactor pressure to a value below 15 bar.

13. A process according to claim 11, wherein liquid reaction product is retained in the unit during the high-speed stop.

14. A process according to claim 11, wherein liquid reaction product is removed from the reactor during the high-speed stop.

15. A process according to claim 11, wherein the provision of CO is stopped after 2-20 minutes.

16. A process according to claim 11, wherein the CO is provided from a storage vessel containing a CO-containing gas with a CO content of at least 80 mol. %, the balance consisting of a gas which is inert under Fischer-Tropsch reaction conditions.

17. A process for carrying out a high-speed stop in a Fischer-Tropsch process which comprises providing a feed comprising CO and H₂ to a fixed bed reactor comprising a Fischer-Tropsch catalyst, the reactor being at reaction temperature and pressure, the gaseous hourly space velocity in the reactor being in the range from 500 to 10000 Nl/l/h, and withdrawing an effluent from the reactor;
wherein the high-speed stop is effected by blocking provision of H₂ to the reactor while providing CO to the reactor, and withdrawing gaseous reactor content from the reactor;
wherein during the high-speed stop CO is added in an amount of 5-80 Nl/l/h; and
wherein the CO is provided from a storage vessel containing a CO-containing gas with a CO content of at least 80 mol. %, the balance consisting of a gas which is inert under Fischer-Tropsch reaction conditions.

18. A process according to claim 17, wherein the gaseous reactor content is removed to reduce the reactor pressure to a value below 15 bar.

19. A process according to claim 17, wherein the provision of CO is stopped after 2-20 minutes.

20. A process according to claim 17, wherein the reactor is a reactor tube with a ratio between length and diameter of at least 50:1.
